(19) United States
(12) Patent Application Publication
(10) Pub. No.: US 2010/0105750 A1
(43) Pub. Date: Apr. 29, 2010

(54) STABLE METRONIDAZOLE GEL FORMULATIONS

(75) Inventors: Brian Aksamit, Shirley, NY (US); Robert J. Anderson, Stoney Brook, NY (US); Kuljit Bhatia, Nesconset, NY (US); Sandhya Goyal, Westbury, NY (US)

Correspondence Address: SCHWEGMAN, LUNDBERG & WOESSNER, P.A., P.O. BOX 2938, MINNEAPOLIS, MN, 55402, US

(73) Assignee: NYCOMED US INC., MELVILLE, NY (US)

(21) Appl. No.: 12/605,008

(22) Filed: Oct. 23, 2009

Related U.S. Application Data
(60) Provisional application No. 61/107,906, filed on Oct. 23, 2008.

(51) Int. Cl. A61K 31/4164; A61P 17/00

(52) U.S. Cl. 514/398

(57) ABSTRACT

The invention provides aqueous gel compositions comprising metronidazole, benzyl alcohol as a solvent, water, and a polyacrylic acid or cellulosic gelling agent, wherein the benzyl alcohol is present in an amount effective to maintain the physical stability of the aqueous gel composition for at least seven days at 5° C. The invention also provides methods of using and methods of preparing the aqueous gel compositions.

### RELATED APPLICATIONS

This application claims priority under 35 U.S.C. §119(e) to U.S. Provisional Patent Application No. 61/107,906, filed Oct. 23, 2008, which is incorporated herein by reference.

### BACKGROUND OF THE INVENTION

Metronidazole, 1-(2-hydroxyethyl)-2-methyl-5-nitroimidazole, is known as an effective drug to treat a variety of disorders, including the treatment of various protozoal diseases. As a topical therapy, metronidazole is useful to treat various skin disorders, including acne rosacea, bacterial ulcers, and perioral dermatitis (see, Borgman, U.S. Pat. No. 4,837,378). Metronidazole has been found to have anti-inflammatory activity when used topically to treat dermatologic disorders (see, Czernielewski, et al., U.S. Pat. No. 5,849,776). Metronidazole may also be used as an intravaginal therapeutic agent for the treatment of bacterial vaginosis (see, Borgman, U.S. Pat. No. 5,536,743).

Compositions containing metronidazole for treatment of dermatologic disorders are available in various forms, including creams, lotions, and gels. One commercially available metronidazole product, Noritate® cream (Dermik Laboratories, Inc.), contains 1% metronidazole, where the insoluble metronidazole is suspended in an opaque cream. A commercially available gel product, Metrogel® topical gel (Galderma Laboratories, Inc.), contains 1% metronidazole, where the metronidazole is solubilized during the gel preparation.

For the treatment of many dermatologic and mucosal disorders, it is often preferable to use a solubilized water-based formulation, such as a gel, rather than a cream, lotion or an ointment. Creams, lotions (typically oil in water emulsions) and ointments (typically petroleum jelly based compositions) are often comedogenic, acnegenic, or are not cosmetically appealing to patients. Also, solubilized active ingredients in topical products are generally more bioavailable than products in which the active ingredient has not been solubilized.

Oil-based cream and ointment metronidazole formulations can provide an advantage over gel-based formulations because oil-based formulations have been found to solubilize a higher concentration of metronidazole. Aqueous-based gel compositions have been limited to lower concentrations of metronidazole because of the poor solubility of metronidazole in water.

Various attempts have been made to increase the solubility of metronidazole in gel formulations. For example, cyclodextrins have been shown to enhance the solubility of various drugs in aqueous solutions. An amphiphilic or lipophilic drug, such as metronidazole, can be partially surrounded by the cyclodextrins, thereby increasing the solubility of the drug in aqueous media. Cyclodextrins have certain disadvantages, however, including cost, limitations of cyclodextrin solubility, incompatibility in certain vehicles, and potential for local and systemic toxicity.

Researchers have also described the use of beta-cyclodextrin (BCD) in combination with metronidazole. Kata and Antal (Acta Pharmaceutica Hungarica, 54:116-122 (1984)) describe an increase in the rate of dissolution of metronidazole when dissolved in a solution containing BCD at 37° C. However, the stability of the BCD/metronidazole solutions is not addressed. Disadvantages of the use of BCD to solubilize drugs such as metronidazole include the relatively low solubility of BCD in water and that BCD is a relatively inefficient solubilizer, particularly for lipophilic or amphiphilic drugs, such as metronidazole. Additionally, cyclodextrins, such as BCD and its derivatives, are expensive and thus drug formulations containing BCD as a solubilizing agent become expensive.

Therefore, a need exists for methods to increase the solubility of metronidazole without the use of cyclodextrins such as BCD. Solubility enhancing agents other than cyclodextrins have been described. For example, Chien (J. Parenteral Science and Technology, 38(1):32-36 (January 1984)) discloses that niacinamide is a solubility enhancing agent that can increase the water solubility of metronidazole. However, current commercial products still have stability problems, such as the crystallization of metronidazole during storage, for example, at below room temperature.

Accordingly, there is a need for stable metronidazole gel formulations that maintain the solubility of metronidazole without the use of expensive solubilizing agents. There is also a need for stable metronidazole gel formulations that maintain the solubility of metronidazole at lower than ambient temperatures during storage.

### SUMMARY

The invention provides an aqueous gel composition comprising about 0.5 wt. % to about 2 wt. % metronidazole, about 0.5 wt. % to about 5 wt. % benzyl alcohol as a solvent, at least about 80 wt. % water, and about 0.25 wt. % to about 2 wt. % of a polyacrylic acid or cellulosic gelling agent; wherein the benzyl alcohol is present in an amount effective to maintain the physical stability of the aqueous gel composition for at least about seven days at 5° C.

It has been unexpectedly discovered that the addition of benzyl alcohol to an aqueous metronidazole gel, without other solubility enhancing agents, provides a significant stabilizing effect to the aqueous gel. For example, metronidazole gels were prepared that were physically stable at 5° C. for more than 32 weeks. These discoveries permit the production of aqueous metronidazole gels where the amount of dissolved metronidazole can be at least about 1 wt. %. At such levels, the metronidazole gels can be effectively used as topical medicaments.

The gelling agent can be a carbomer gelling agent or a cellulosic gelling agent. The pH of the gel can be about 4 to about 5, and the viscosity can be in the range of about 500 to about 32,000 cps. In some embodiments, the gel composition does not comprise a cosolvent and/or a solubility enhancing agent other than benzyl alcohol. In some embodiments, the gel is free of, or substantially free of, niacinamide, niacin, and/or cyclodextrins.

The gel composition can be clear and/or colorless. In some embodiments, the benzyl alcohol is present in an amount effective to maintain the physical stability of the aqueous gel composition for at least about 28 days at 5° C. In other embodiments, the gel composition can be physically stable for at least about 60 days, at least about 200 days, or at least about 32 weeks, at 5° C.

The invention further provides methods to treat a dermatologic disorder comprising topically applying to the site of the disorder an effective amount of an aqueous gel composition described herein. The application can be once daily, or more than once daily, for example, two or three applications daily. The disorder can be an inflammatory disorder, for example, rosacea. The invention also provides a kit for treating such disorders. The kit can include a container, such as a jar or tube, and optionally instructions for using the composition.

The invention additionally provides methods to prepare an aqueous gel composition that contains metronidazole and benzyl alcohol. The method can include combining metronidazole, benzyl alcohol, and a gelling agent in an aqueous solution, where the gel composition contains benzyl alcohol at a concentration greater than about 0.5 wt. %. The amount of benzyl alcohol in the resulting aqueous gel is sufficient to provide a dissolved concentration of metronidazole of greater than 0.5 wt. % at a temperature of 5° C. for greater than six, seven, or eight days. The invention further provides for the use of a stable metronidazole gel for the treatment of skin disorders in a mammal, such as a human, and for the use of a stable metronidazole gel for the manufacture of a medicament useful to treat skin disorders.

The invention also provides a method for increasing the solubility of metronidazole in an aqueous gel composition comprising combining metronidazole, benzyl alcohol, and a gelling agent in an aqueous fluid, wherein the amount of benzyl alcohol in the fluid is at least 0.5 wt. % and the amount of metronidazole in the fluid is at least about 0.75 wt. %, for example, 1.0 wt. %.

### DETAILED DESCRIPTION

The invention provides a stable aqueous gel containing metronidazole and benzyl alcohol. Benzyl alcohol has been found to be surprisingly effective at solubilizing metronidazole in an aqueous gel, without the need for a secondary solubilizing agent. A lack of gel stability (e.g., crystallization of the solubilized metronidazole from the gel) is a common problem with many known metronidazole gel formulations. The use of benzyl alcohol in gel compositions described herein provides significant stability such that metronidazole crystals do not form in the gel, even after more than six months of storage at a temperature of 5° C.

The invention thus provides an aqueous gel that includes metronidazole and benzyl alcohol, wherein the solution is free of crystal or precipitate formation when stored for one week, and typically longer, at 5° C. Use of the aqueous gel provides a method of reducing the inflammation in an inflammatory skin condition, such as rosacea.

Beta-cyclodextrin (BCD) is a complexing agent and is widely known to solubilize a variety of compounds. BCD has a relatively low solubility in water and is a relatively inefficient solubilizer, particularly for lipophilic or amphiphilic drugs such as metronidazole. Additionally, BCD is expensive and has a potential for local or systemic toxicity. Chang et al. (U.S. Pat. No. 6,881,726) describe an aqueous metronidazole gel where the metronidazole is solubilized using BCD in combination with a secondary solubilizer (niacin or niacinamide), in an attempt to maintain the solubility of the metronidazole. Chang et al. define “stable” solutions of metronidazole as showing no crystals or precipitate from solution when stored at refrigerated temperatures of 5° C. for at least 7 days.

Analysis of Metrogel® 1% topical gel, which contains BCD and niacinamide, showed that the commercial product is less stable than the metronidazole gels disclosed herein. Metrogel® 1% topical gel, stored at of 5° C. for 48 days, formed metronidazole crystals, which increased in size and number over time. Formation of crystals can be determined by storing a gel in a glass jar in the dark at a temperature of interest, for example, the standard temperature of refrigerated storage. Visual inspection and/or microscopic (400×) analysis was used to inspect the stored gels. A 1% metronidazole gel containing benzyl alcohol did not show crystal formation or precipitation even after 32 weeks of storage at 5° C.

The International Journal of Pharmaceutical Compounding (Vol. 8, No. 4, July/August 2004, page 300) disclosed a gel formulation containing 2% metronidazole. This formulation, however, contains metronidazole in a suspended form, rather than in a solubilized form, in the gel base. The compositions described herein are more effective at a lower concentration (1%) of metronidazole than the suspended 2% formulation because a solubilized drug penetrates the epidermis more effectively than a suspended drug. Accordingly, the metronidazole gel formulations described herein containing benzyl alcohol provide significant advantages over currently known formulations.

### DEFINITIONS

As used herein, certain terms have the following meanings. All other terms and phrases used in this specification have their ordinary meanings as one of skill would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

The term “and/or” means any one of the items, any combination of the items, or all of the items with which this term is associated.

The singular forms “a,” “an,” and “the” include plural reference unless the context clearly dictates otherwise. Thus, for example, a reference to “a composition” includes a plurality of such compositions, so that a composition X includes a plurality of compositions X.

The term “about” can refer to a variation of ±5%, ±10%, or ±20% of the value specified. For example, “about 50” percent can in some embodiments carry a variation from 45 to 55 percent. For integers or integer ranges, the term “about” can include one or two integers greater than and/or less than a recited integer.

All concentrations or amounts of ingredients referred to in this specification are % w/w, unless otherwise indicated.

As used herein, the term “stable” refers to physical, rather than chemical, stability. The metronidazole solutions and gels of the invention that contain benzyl alcohol are physically stable. For example, substantially no crystal or precipitate from solution is observed, when stored at refrigerated temperatures of 5° C. for at least 7 days. Accordingly, the physical stability of a gel refers to its property of maintaining the solubility of an active ingredient, such as metronidazole, such that the active ingredient does not crystallize or precipitate from the gel upon storage, such as at reduced temperatures, for prolonged periods of time.

The term “solubility enhancing agent” or “solubility enhancer” refers to a chemical compound that, when present in solution in a solvent, increases the solubility of a second chemical compound, such as an active ingredient (e.g., metronidazole), in the solvent, but which chemical compound is not itself a solvent for the second chemical compound. Examples include Poloxamer 182, Poloxamer 407, Polysorbate 20, Polysorbate 40, Polysorbate 80, α-cyclodextrin, β-cyclodextrin, dioctyl sulfosuccinate, and PEG 200.

As used herein, the terms “solvent” and “cosolvent” can refer to an organic solvent that dissolves metronidazole. With respect to this disclosure, benzyl alcohol can be a solvent wherein propylene glycol is optionally not considered a cosolvent, but can be considered a diluent or carrier.

The term “carbomer” refers to a cross-linked polymer of acrylic acid. An example of a carbomer is Carbomer 940, which is a high molecular weight polymer of acrylic acid cross-linked with allyl ethers of pentaerythritol. Many other carbomers are known in the art and may be used in compositions of the invention.

The terms “effective amount” or “therapeutically effective amount” refer to an amount of an active agent or an amount of a gel formulation that will exhibit one or both of an antimicrobial and optionally an anti-inflammatory effect, when applied to an infected or inflamed area of the skin. A single application of the formulations described herein may be sufficient, or the formulations may be applied repeatedly over a period of time, such as once per day, or several times a day, for a period of days or weeks. The duration of treatment will vary with the severity of the condition being treated, the stage of advancement of the condition, the age of the patient, and the type and concentration of the formulation being applied. Appropriate amounts in any given instance will be readily apparent to those skilled in the art and can be determined by a treating medical practitioner, such as a physician.

The term “anti-inflammatory effect” means a reduction in one or more of the symptoms of erythema (redness), edema (swelling), pain or pruritus, which are characteristic of inflammatory skin conditions, such as rosacea.

Rosacea is a chronic inflammatory skin disorder characterized by four distinct clinical stages predominantly affecting the central aspect of the face. The first clinical evidence of rosacea is frequent and intense vasodilation or flushing. Most patients progress to a vascular stage characterized by an erythema that can persist for hours or days after a triggering event. Many patients remain stabilized at this stage, while some patients progress to an inflammatory stage characterized by a symmetrical array of papules and pustules, in addition to the persistent erythema. The inflammatory stage can often become a chronic condition. Some patients, mostly male, can progress to the final stage characterized by a distinctive hyperplasia or swelling, especially of the nose. Rosacea is a very visible skin condition that has a strong impact on the quality of life of the patient.

### Metronidazole Gels

The invention provides stable metronidazole gels that contain effective amounts of benzyl alcohol. Benzyl alcohol has been surprisingly found to substantially increase the stability of aqueous metronidazole gels. Accordingly, the invention provides an aqueous gel composition that includes about 0.1 wt. % to about 5 wt. % metronidazole, about 0.5 wt. % to about 5 wt. % benzyl alcohol as a solvent, water, and a polyacrylic acid or cellulosic gelling agent; wherein the benzyl alcohol is present in an amount effective to maintain the physical stability of the aqueous gel composition for at least about seven days at 5° C. For example, the aqueous gel can include about 0.5 wt. % to about 2 wt. % metronidazole, about 0.5 wt. % to about 5 wt. % benzyl alcohol as a solvent, at least about 80 wt. % water, and a polyacrylic acid or cellulosic gelling agent present in about 0.25 wt. % to about 2 wt. %.

In one embodiment, the gel composition includes about 0.5 wt. %, about 0.75 wt. %, about 1 wt. %, or about 1.5 wt. % of metronidazole. The amount of benzyl alcohol effective to maintain the physical stability of the aqueous gel composition for at least about seven days at 5° C. can also be about 1 wt. % to about 3.5 wt. %. The gel composition can include an amount of benzyl alcohol effective to maintain the physical stability of the aqueous gel composition for at least 7 days, 28 days, 60 days, 200 day, or 32 weeks, at 5° C. In some embodiments, the benzyl alcohol can be present in about 1 wt. %, about 1.5 wt. %, about 2 wt. %, about 2.5 wt. %, about 3 wt. %, or about 3.5 wt. %, or in a range between any two of the aforementioned values. In some embodiments, the gel composition does not comprise a cosolvent. For example, the gel composition can include benzyl alcohol but does not include a solubility enhancing agent.

Any gelling agent that is water-dispersible and forms an aqueous gel of substantially uniform consistency is suitable for use in the compositions of the invention, so long as the gelling agent does not substantially interfere with the water solubility of metronidazole or with the therapeutic efficacy of the gel. “Substantially interfere” means that the inclusion of the agent decreases the solubility of metronidazole to 0.75 wt. % or less in aqueous solution. Suitable gelling agents include polyacrylic acid gelling agents and/or cellulosic gelling agents. One gelling agent can be hydroxyethylcellulose (Natrosol™, Hercules Inc., Wilmington, Del.). Examples of other suitable gelling agents include carboxyvinyl polymers, such as Carbopol® 934, 940, or 941 (Noveon, Inc., Akron, Ohio).

The polyacrylic acid gelling agent can be a carbomer gelling agent, such as Carbomer 940. The carbomer gelling agent can be present in about 0.2-1 wt. %, for example, about 0.25 wt. % or about 0.5 wt. %. In some embodiments, the polyacrylic acid gel compositions include about 2.5-3.5 wt. % of benzyl alcohol. In certain specific embodiments, the composition includes about 3 wt. % of benzyl alcohol. The pH of the carbomer composition can be about 4 to about 6. In some embodiments, the pH will be about 4.5 to about 5.6, about 4.6 to about 4.9, or about 4.7. The viscosity of the carbomer composition can be about 500 cps to about 32,000 cps, about 500 cps to about 15,000 cps, about 10,000 cps to about 15,000 cps, or about 26,000 cps to about 32,000 cps, depending on the amount of carbomer used in the formulation.

The cellulosic gelling agent can be a hydroxyalkyl cellulose gelling agent, for example, hydroxyethyl cellulose (HEC) or hydroxymethyl cellulose (HMC). The cellulosic gelling agent can be present in about 1-3 wt. %, for example, about 1 wt. %, about 1.25 wt. %, about 1.5 wt. %, or about 2 wt. %. In some embodiments, the cellulosic gel compositions include about 2-3 wt. % of benzyl alcohol. In certain specific embodiments, the composition includes about 2.5 wt. % of benzyl alcohol. The pH of the cellulosic gel composition can be about 4 to about 6. In some embodiments, the pH will be about 5.0 to about 5.5, about 5.2 to about 5.5, or about 5.3, or about 5.4. The viscosity of the cellulosic gel composition can be about 500 cps to about 12,000 cps, about 6,000 cps to about 10,000 cps, or about 7,000 cps to about 9,000 cps, for example, about 8,000 cps, depending on the amount of cellulosic gelling agent used in the formulation.

In one embodiment, the composition includes about 1% metronidazole, about 3% benzyl alcohol, about 0.5% Carbomer 940, about 3% propylene glycol, about 0.05% edetate disodium, about 0.1% parabens, about 0.04% sodium hydroxide, and at least about 90% water; wherein the benzyl alcohol is present in an amount that is effective to maintain the physical stability of the aqueous gel solution for at least seven days at 5° C. In another embodiment, the gel composition includes about 1% metronidazole, about 2.5% benzyl alcohol, about 1.25% hydroxyethyl cellulose, about 3% propylene glycol, about 0.05% edetate disodium, about 0.1% parabens, and at least about 90% water; wherein the benzyl alcohol is present in an amount that is effective to maintain the physical stability of the aqueous gel solution for at least seven days at 5° C. Of course, any of these compositions can be stable for significantly longer periods of time as well. The composition can optionally include other inactive ingredients that do not substantially interfere with the solubility of metronidazole.

The compositions of the invention can also be free of, or substantially free of, aqueous solubility-enhancing agents other than benzyl alcohol. In some embodiments, the aqueous gel composition does not include certain agents, such as a cyclodextrin, niacinamide, or niacin.

### Method of Preparing Metronidazole Gels

The aqueous gels of the invention may be made in any way that results in a stable metronidazole concentration of greater than 0.75%, preferably of 1.0%, or higher. Preferably, the solubility enhancers and the metronidazole are combined in water, or a water-based solution, before the addition of a gelling agent, or before gelling of the solution occurs. Preferably, the solubility enhancers are dissolved in water before addition of the metronidazole.

Accordingly, the invention provides methods for preparing an aqueous gel composition that contains metronidazole. Such methods can include combining metronidazole, benzyl alcohol, and a gelling agent in an aqueous solution, where the gel composition contains benzyl alcohol at a concentration greater than about 0.5 wt. %, for example, about 2-3.5 wt. %, and the amount of benzyl alcohol in the resulting aqueous gel is sufficient to provide a dissolved concentration of metronidazole of greater than 0.5 wt. % (e.g., about 1 wt. %) at a temperature of 5° C. for greater than seven days, and typically substantially longer periods of time.

The gels described herein can generally be made by standard gel preparation techniques. For example, purified water can be heated and mixed or agitated with desired amounts of edetate disodium, benzyl alcohol, and metronidazole until the solids are dissolved. A gelling agent, such as Carbomer 940 or hydroxyethyl cellulose, can be slowly added until homogeneous to provide a first mixture. Propylene glycol can be added, followed by preservatives, such as methylparaben and/or propylparaben.

Aqueous sodium hydroxide can be added if a carbomer is used as the gelling agent, with stirring. Continued stirring provides a clear, smooth gel. The pH can be adjusted, if necessary, by slow addition of a dilute aqueous 1.5% sodium hydroxide if the pH is below about 4. Purified water (q.s. 100%) can then be added with stirring to provide the final appropriate concentration. The batch can be strained directly into storage containers. The gel should be protected from light.

Thus, physically stable aqueous solutions of metronidazole at concentrations greater than about 0.75% can be obtained without the presence of cyclodextrins. Cyclodextrins can be toxic to humans, at certain levels. The compositions described herein are especially effective for treating topical dermatologic conditions, such as rosacea, that may be worsened by irritating adjuvants or solubility enhancing agents present in known commercial formulations.

The stable aqueous metronidazole solutions can have a concentration of metronidazole greater than 0.75 wt. %. Preferably, the concentration of metronidazole in the composition is about 1.0%. The concentration of metronidazole in the aqueous gel may be even higher, such as 1.25%, 1.5%, 2.0%, or 2.5%, or more. At a level of 1% or higher of metronidazole, the aqueous gel may be effectively used as a topical formulation for therapeutically purposes, as described herein.

The gel formulations are typically non-tacky, fast-drying, and cosmetically elegant. The gel formulations are physically stable at 5° C. (refrigerator temperature) and/or at room temperature conditions for at least 7 days. No crystal formation or precipitation is observed after one week at 5° C. for the compositions of the invention.

The invention also provides a method for increasing the solubility of metronidazole in an aqueous gel composition, as well as a method for maintaining the stability of metronidazole in an aqueous gel composition. The methods include combining metronidazole, benzyl alcohol, and a gelling agent in an aqueous fluid, wherein the amount of benzyl alcohol in the fluid is at least 0.5 wt. % and the amount of metronidazole in the fluid is at least about 0.5 wt. %, about 0.75 wt. %, or about 1 wt. %.

### Methods of Treating Dermatological Disorders

The aqueous gels may be used for the topical treatment of dermatologic disorders that are responsive to therapy with metronidazole. In accordance with the methods described herein, a stable aqueous gel containing metronidazole at a concentration higher than about 0.75 wt. %, preferably about 1 wt. % or higher, is topically applied to skin in need of such therapy. The therapeutic method may be used to treat any disorder that is responsive, or potentially responsive, to metronidazole therapy. Examples of disorders that can be suitably treated include inflammatory lesions on the skin, diabetic foot ulcers, and certain infectious diseases that may be treated topically. In one embodiment, the method includes treating rosacea.

At concentrations of about 1% or higher, the application of the metronidazole gel can be carried out once per day, or more than once per day, as directed by a medical professional. The gel can be applied on a daily basis, or one or more times per day, for a time sufficient to produce an amelioration or a cure of a disorder. In certain chronic disorders, the solution may be applied one or more times daily for a prolonged period to prevent worsening of the disorder.

In another embodiment, a kit is provided for the topical treatment of skin or mucosal disorders. The kit can include a jar, tube, syringe, or other container suitable for holding an aqueous metronidazole solution or gel as described herein, and instructions for applying the solution topically to affected areas, for example, to skin or a mucosal surface. The metronidazole solution can have a concentration of metronidazole of about 1% or higher and the instructions may call for applying the metronidazole solution to affected areas, for example, once daily. The container may be packaged within a box that includes labeling information, and/or additional information, such as instructions for using the composition.

The invention thus provides methods for treating dermatologic disorders comprising topically applying to the site of the disorder an effective amount of an aqueous gel composition as described herein. The composition can include about 0.5 wt. % to about 2 wt. % metronidazole, about 0.5 wt. % to about 5 wt. % benzyl alcohol as a solvent, at least about 80 wt. % water, and a polyacrylic acid or cellulosic gelling agent present in about 0.25 wt. % to about 2 wt. %. The benzyl alcohol is present in an amount effective to maintain the physical stability of the aqueous gel composition for at least seven days at 5° C. The application can be once daily, or it can be more than once per day, as directed by a medical practitioner. The condition or disorder can be an inflammatory disorder, such as rosacea.

The following Examples are intended to illustrate the above invention and should not be construed as to narrow its scope. One skilled in the art will readily recognize that the Examples suggest many other ways in which the invention can be prepared and practiced. It should be understood that numerous variations and modifications may be made while remaining within the scope of the invention.

### EXAMPLES

### Example 1

### Stable Metronidazole Gel Formulations

Metronidazole (1%) gels have been known to be unstable when stored for prolonged periods of time, due to the limited solubility of the active in gel compositions. Some metronidazole gel formulations form crystals during warehouse storage, for example, at temperatures of about 15-19° C., or during refrigerated storage, for example, at temperatures of about 5° C. These products then have lower concentrations of solubilized active and therefore provide diminished therapeutic efficacy. This Example describes the discovery of formulations that maintain the solubility of metronidazole in gel compositions by including specific solvent systems.

The solubility of metronidazole in solution has been shown to be pH dependent under various conditions. Certain metronidazole gels (e.g., containing more than 95% water) have been developed wherein the pH is adjusted to keep the metronidazole in solution. However, these gel compositions are unstable and can provide only borderline metronidazole solubility, particularly when stored at a reduced temperature. Crystallization of the metronidazole active can often be observed. Such pH adjusted products showed crystals when stored in a warehouse at 15-19° C. The formulations described herein were designed to avoid such crystal formation.

Experimental design. The gel formulations described herein were found to maintain the metronidazole Active Pharmaceutical Ingredient (API) in solution, even upon refrigeration (e.g., at 4-5° C.) for more than 5 days. These formulations provide an aqueous gel with viscosity similar to the reference listed drug (RLD), Metrogel® 1% topical gel.

More than 70 prototype formulations were prepared and evaluated under a variety of conditions. Each of the gels was evaluated for stability (e.g., the ability to maintain API solubility and the lack of metronidazole crystal formation) at various temperatures, including 5° C., 10° C. and 15° C., for prolonged periods of time. Acrylic polymers, such as the carbopols (e.g., carbomers), and cellulosic polymers, such as hydroxyethyl cellulose, were evaluated at various concentrations to determine their effect on viscosity, solubility and crystal formation. The ingredients of two specific formulations are provided in Table A.

TABLE A
Two Specific Examples of 1% Metronidazole Gel Formulations

| | | | Carbomer | HEC |
|---|---|---|---|---|
| | | RLD | Formula I | Formula II |
| | Ingredient | (% w/w) | (% w/w) | (% w/w) |
| 1 | Purified Water USP | QS | QS | QS |
| 2 | Edetate Disodium USP | 0.05 | 0.05 | 0.05 |
| 3 | Benzyl alcohol NF | — | 3.00 | 2.50 |
| 4 | Metronidazole USP | 1.00 | 1.00 | 1.00 |
| 5 | Carbomer 940 NF | — | 0.5 | — |
| 6 | Hydroxyethyl cellulose | 1.25 | — | 1.25 |
| 7 | Propylene Glycol USP | 3.00 | 3.00 | 3.00 |
| 8 | Methylparaben NF | 0.15 | 0.08 | 0.08 |
| 9 | Propylparaben NF | 0.06 | 0.02 | 0.02 |
| 10 | Purified Water USP | — | 0.40 | — |
| 11 | Sodium Hydroxide NF | — | 0.04 | — |
| 12 | Phenoxyethanol | 0.70 | — | — |
| 13 | Beta - Cyclodextrins | 1.00 | — | — |
| 13 | Nicotinamide | 1.25 | — | — |
| | (Niacinamide) | | | |

Results and discussion. Gels were prepared on a 0.5-1 kg scale. It was expected that the addition of an organic solubility enhancing agent to the metronidazole gel formulations would increase the metronidazole solubility and decrease crystal formation. Water miscible solubility enhancing agents, for example, poloxamers, polyethylene glycols, cyclodextrins, and the like, were expected to be especially suitable for providing metronidazole stability in aqueous gel formulations, such as 1 wt. % metronidazole gels that include greater than about 90 wt. % water.

However, it was surprisingly discovered that benzyl alcohol, which is only moderately water soluble (4 g/100 mL), provided superior stability properties over other solvents with higher water solubility. Only benzyl alcohol, when added to the metronidazole formulations, prevented crystal formation under the reduced temperature evaluation conditions. It was also surprisingly discovered that higher weight percentages of benzyl alcohol (e.g., greater than about 5 or 10 wt. %) did not improve the quality (e.g., stability) of the gel formulations. Accordingly, it was unexpectedly found that only a narrow concentration range of benzyl alcohol in the metronidazole gel formulations provided the increased stability.

A standard metronidazole gel was prepared to include 0.5 wt. % polyacrylic acid, 1 wt. % metronidazole, and water. The stability of the gels was evaluated by including an organic solvent in which metronidazole is highly soluble. Only benzyl alcohol prevented crystal formation when the gels were stored at 5° C., 10° C., and 15° C. for prolonged periods of time. It was determined that too little benzyl alcohol decreased the stability of the formulation, and also that concentrations of benzyl alcohol greater than about 5 wt. % were deleterious to the clarity of the gel formulation. Concentrations of benzyl alcohol from about 0.5 wt. % to about 3.5 wt. % provided significantly increased stability over formulations without benzyl alcohol (Table 1).

TABLE 1
Gel Formulation with 0.5 wt. % Carbomer
and 1 wt. % Metronidazole

| Conc. Benzyl | Crystals @ | Crystals @ | Crystals @ |
|---|---|---|---|
| Alcohol % | 5° C. | 10° C. | 15° C. |
| 0.5 | 4 days | None 28 days | None 28 days |
| 1.0 | 4 days | None 28 days | None 28 days |
| 1.5 | None 28 days | None 28 days | None 28 days |
| 2.0 | None 28 days | None 28 days | None 28 days |
| 2.5 | None 28 days | None 28 days | None 28 days |
| 3.0 | None 28 days | None 28 days | None 28 days |
| 3.5 | None 28 days | None 28 days | None 28 days |

It was determined that, if used alone, at least about 1.0 wt. %, or at least about 1.5 wt. %, of benzyl alcohol is sufficient to maintain metronidazole in solution and to prevent crystal formation during prolonged refrigeration. Inclusion of benzyl alcohol in excess of about 3.5% resulted in decreased gel clarity (e.g., increased cloudiness), indicating a decrease in gel stability beyond such levels.

Solubility Enhancing Agent Analysis. For solubility enhancing agent studies, the concentrations of benzyl alcohol evaluated included 1, 1.5, 1.75, 2.0, and 2.5 wt. %. Several specific data are provided in Table 2.

TABLE 2
Solubility Enhancing Agent Evaluation

| | | Initial | Crystals @ | Crystals @ | Crystals @ |
|---|---|---|---|---|---|
| ID | Agents | Gel | 5° C. | 10° C. | 15° C. |
| #1 | 1% Benzyl Alcohol | Clear, | None 12 days | None 12 days | None 12 days |
| | 1% Poloxamer 407 | colorless | | | |
| | Carbomer | gel | | | |
| #2 | 1% Benzyl Alcohol | Clear, | None 12 days | None 12 days | None 12 days |
| | 0.2% Polysorbate 40* | colorless | | | |
| | Carbomer | gel | | | |
| #3 | 1% Benzyl Alcohol | Clear, | None 12 days | None 12 days | None 12 days |
| | 1% Polysorbate 20 | colorless | | | |
| | Carbomer | gel | | | |
| #4 | 1.5% Benzyl Alcohol | Clear gel | None 7 days | None 7 days | None 7 days |
| | 1% Polysorbate 20 | | | | |
| | HEC | | | | |
| #5 | 1% Benzyl Alcohol | Clear, | None 26 days | None 26 days | None 26 days |
| | 0.2% Polysorbate 40* | colorless | | | |
| | Carbomer | gel | | | |

*IIG (inactive ingredient) limit for Polysorbate 40 is 0.2%.

In addition to the data of Table 2, a formulation prepared using 1% benzyl alcohol and 1% Polysorbate 80 in a carbomer gelling agent provided a clear colorless gel that prevented crystal formation reliably for only about one day at less than ambient temperatures. A formulation prepared using 1% benzyl alcohol and 1% Polysorbate 20 in HEC also provided a clear, although not colorless, gel.

Other combinations of gel solubility enhancing agent systems prepared included benzyl alcohol 2% and Polysorbate 20, 1% (HEC or Carbomer); benzyl alcohol 2.5% and Polysorbate 20, 1% (Carbomer); benzyl alcohol 2.0% and Polysorbate 20, 0.5% (HEC or Carbomer); benzyl alcohol 1.5% and Polysorbate 20, 1% (Carbomer); benzyl alcohol 2.0% and Polysorbate 40, 0.2% (Carbomer); benzyl alcohol 2.5% and Polysorbate 40, 0.2% (Carbomer); benzyl alcohol 1.75% and Polysorbate 40, 0.2% (Carbomer); and benzyl alcohol 1.5% and Polysorbate 40, 0.2% (Carbomer); each of which provided cloudy gels, which are less desirable for therapeutic formulations. Cloudiness also indicates physical instability.

The various carbomer and HEC gels prepared with benzyl alcohol and Poloxamers 182 and 407, respectively, were hazy at room temperature (˜23° C.), except for Formulation #1 (Table 2). HEC formulations exhibited a higher tolerance for Polysorbate 20 when used in combination with benzyl alcohol (up to about 1.5 wt. %). For example, the concentration of Polysorbate 20 could be increased to 1 wt. % with 1.5 wt. % benzyl alcohol (Formulation #4), without causing the gel to become cloudy.

It was determined that gel formulations with 1 wt. % Polysorbate 20 and/or 0.2 wt. % Polysorbate 40 suitably maintain the active in solution when used in conjunction with benzyl alcohol (Formulations #2-5). Formulations with a combination of benzyl alcohol and Polysorbate 20 at concentrations of 1 wt. % provided clear colorless carbomer-based gels (Formulation #3). Increasing the concentration of benzyl alcohol produced cloudy gels. These benzyl alcohol-containing formulations (with or without co-solvents) did not form crystals in gels stored at any of the three study temperatures for at least seven days. However, none were as effective as benzyl alcohol alone as a solubility enhancing agent.

A solubility enhancing agent analysis was carried out on the HEC-based metronidazole gel samples for the successful solubility enhancing agent used with carbomer-based gels, following the methods used to prepare the data of Table 2.

TABLE 3
Solubility Enhancing Agent Evaluation for HEC-based Gels

| | | Crystals @ | Crystals @ | Crystals @ |
|---|---|---|---|---|
| ID | Agents | 5° C. | 10° C. | 15° C. |
| #6 | 1% Benzyl Alcohol | None 11 days | None 11 days | None 11 days |
| | l % Poloxamer 407 | | | |
| #7 | 1% Benzyl Alcohol | 1 at 10 days | None 11 days | None 11 days |
| | 0.2% Polysorbate 40 | | | |
| #8 | 1% Benzyl Alcohol | None 8 days | None 8 days | None 8 days |
| | 1% Polysorbate 20 | | | |

While the HEC-based gels prepared using solubility enhancing agents or surfactants provided increased stability in terms of reduced crystal formation, they did not maintain the desired clarity and color properties compared to formulations containing only benzyl alcohol. The gel containing poloxamer 407 (Formulation #6) became cloudy over time at room temperature, and the Polysorbate 40 and Polysorbate 20 formulations (Formulations #7 and #8, respectively) did not maintain suitable gel clarity. Accordingly, benzyl alcohol alone as a solubility enhancing agent for metronidazole in an aqueous gel provides a surprising increase in stability compared to other solubility enhancing agents.

### Gel Stability at Elevated Temperature

Samples of carbomer and HEC based gels were stored at 40° C. (104° F.) and 75% relative humidity (RH) to generate chemical stability data. At 9 months of storage at 25° C./60% RH and 6 months at 40° C./75% RH, the physiochemical stability results of the Carbomer Formula I and Galderma's Metrogel® 1% topical gel showed metronidazole at approximately 100% LC. Other properties, such as viscosity and pH, remained substantially constant over time and within specifications.

Polymer Evaluation. Metronidazole formulations containing the gelling agents Carbomer (0.5 wt. % and 0.25 wt. %) and HEC (1.25 wt. %) were separately prepared. Each formulation exhibited suitable gel stability. The formulations containing 0.25 wt. % carbomer and 1.25 wt. % HEC closely matched the release profile and viscosity of Metrogel® topical gel. Various concentrations of benzyl alcohol were added to study the suitability of the gelling agent, as illustrated below in Tables 3 and 4.

TABLE 3
Benzyl Alcohol Conc. Evaluated in
0.25 wt. % Carbomer Formulations

| Benzyl Alcohol | Crystals @ | Crystals @ | Crystals @ |
|---|---|---|---|
| (wt. %) | 5° C. | 10° C. | 15° C. |
| 0.5 | 1 day | 1 day | 1 day |
| 1.0 | 1 day | 1 day | 1 day |
| 1.25 | None 12 days | None 12 days | 5 days |
| 1.50 | 5 days | None 12 days | None 12 days |
| 2.0 | None 21 days | None 21 days | None 21 days |

TABLE 4
Benzyl Alcohol Conc. Evaluated in 1.25 wt. % HEC Formulations

| Benzyl Alcohol | Crystals @ | Crystals @ | Crystals @ |
|---|---|---|---|
| (wt. %) | 5° C. | 10° C. | 15° C. |
| 2.0 | 8 days | None 21 days | None 21 days |
| 2.5 | None 21 days | None 21 days | None 21 days |
| 3.0 | None 21 days | None 21 days | None 21 days |

The results shown in Tables 3 and 4 demonstrate that the metronidazole gel formulations containing 0.25 wt. % carbomer and at least about 1.25 wt. % benzyl alcohol, and 1.25 wt. % HEC and at least about 2.0 wt. % benzyl alcohol, are substantially stable under typical warehouse storage conditions.

Additional testing that Carbomer Formulation I is stable at 5° C. for more than two years.

### Carbomer-Based Gels

The 0.5% Carbomer-based gel was then further investigated by repeating, on kilogram scale, experiments that provided the results shown in Table 1. Tables 5-7 show the gel stability study results for batches of 1% metronidazole gel prepared at 2 kg, 2 kg, and 40 kg scales, respectively.

TABLE 5
Carbomer-based Gels (0.5%): 2 Kilo Scale Analysis #1

| Benzyl Alcohol | Crystals @ | Crystals @ | Crystals @ |
|---|---|---|---|
| (wt. %) | 5° C. | 10° C. | 15° C. |
| 1.5 | 8 days | 14 days | None 28 days |
| 2.0 | 8 days | 28 days | None 28 days |
| 2.5 | 12 days | None 28 days | None 28 days |
| 3.0 | None 12 days | None 28 days | None 28 days |
| 3.5 (cloudy) | None 12 days | None 28 days | None 28 days |

TABLE 6
Carbomer-based Gels (0.5%): 2 Kilo Scale Analysis #2

| Benzyl Alcohol | Crystals @ | Crystals @ | Crystals @ |
|---|---|---|---|
| (wt. %) | 5° C. | 10° C. | 15° C. |
| 1.5 | 6 days | 28 days | None 28 days |
| 2.0 | 10 days | 28 days | None 28 days |
| 2.5 | 10 days | None 28 days | None 28 days |
| 3.0 | None 12 days | None 28 days | None 28 days |
| 3.5 (cloudy) | None 12 days | None 28 days | None 28 days |

TABLE 7
Carbomer-based Gels (0.5%): 40 Kilo Scale Analysis

| Benzyl Alcohol | Crystals @ | Crystals @ | Crystals @ |
|---|---|---|---|
| (wt. %) | 5° C. | 10° C. | 15° C. |
| 3.0 | None 28 days | None 28 days | None 28 days |

The results shown in Tables 5-7 clearly show the scalability of the highly stable 0.5% carbomer-based 1% metronidazole formulation containing benzyl alcohol, even at production (40 kg) scale. Formulations containing 3.0 wt. % of benzyl alcohol were found to be particularly stable.

### HEC-Based Gels

The HEC-based gels were also evaluated at larger scales. Various 1.25 wt. % HEC-based gels were prepared at kilogram scale and the concentration of benzyl alcohol was evaluated for its effect on preventing metronidazole crystal formation at reduced temperatures.

TABLE 8
HEC-based Gels (1.25%): 2 Kilo Scale Analysis #1

| Benzyl Alcohol | Crystals @ | Crystals @ | Crystals @ |
|---|---|---|---|
| (wt. %) | 5° C. | 10° C. | 15° C. |
| 1.5 | 10 days | 13 days | 21 days |
| 2.0 | 14 days | 14 days | 28 days |
| 2.5 | None 28 days | None 28 days | 28 days |
| 3.0 | 21 days | 21 days | None 28 days |
| 3.5 (hazy) | 21 days | 21 days | None 28 days |

TABLE 9
HEC-based Gels (1.25%): 2 Kilo Scale Analysis #2

| Benzyl Alcohol | Crystals @ | Crystals @ | Crystals @ |
|---|---|---|---|
| (wt. %) | 5° C. | 10° C. | 15° C. |
| 1.5 | 10 days | 13 days | 21 days |
| 2.0 | 12 days | 13 days | 21 days |
| 2.5 | 14 days | None 28 days | 21 days |
| 3.0 | None 28 days | 21 days | None 28 days |
| 3.5 (hazy) | 12 days | None 28 days | None 28 days |

TABLE 10
HEC-based Gels (1.25%): 40 Kilo Scale Analysis

| Benzyl Alcohol | Crystals @ | Crystals @ | Crystals @ |
|---|---|---|---|
| (wt. %) | 5° C. | 10° C. | 15° C. |
| 2.5 | None 28 days | None 28 days | None 28 days |

The results shown in Tables 8-10 clearly show the scalability of the 1.25% HEC-based 1% metronidazole formulations containing benzyl alcohol, even at production (40 kg) scale. Formulations containing 2.5 wt. % benzyl alcohol were found to be especially stable.

Physical Properties. The physical properties of the gels, including viscosity and pH, were measured and were compared to a commercial metronidazole gel product. The data is shown below in Table 12.

TABLE 12
Physical Properties of Various Gel Formulations

| | | Viscosity | pH |
|---|---|---|---|
| ID | Metronidazole Formulation | (CPS) | (neat) |
| A | 0.5% Carbomer | 28,833 | 4.73 |
| | 2.0% Benzyl Alcohol | | |
| B | 0.25% Carbomer | 12,000 | 5.55 |
| | 2.0% Benzyl Alcohol | | |
| C | 1.25% HEC | 8,000 | 5.40 |
| | 2.5% Benzyl Alcohol | | |
| D | 1.25% HEC | 8,000 | 5.29 |
| | 3.0% Benzyl Alcohol | | |
| | Metrogel ® Topical Gel | 7,500 | 6.03 |
| | (1.25% HEC) | | |
| | Metrogel ® Topical Gel, | 11,667 | 5.98 |
| | 2^{nd} Lot (1.25% HEC) | | |

Each benzyl alcohol-containing metronidazole gel formulation provided suitable viscosity for topical formulations, and provided the added benefit of a relatively low pH, which can increase the effectiveness of the gel toward certain skin conditions, for example, rosacea.

Large Scale Preparations. A large scale batch of 0.5% Carbomer Formula I was manufactured for further evaluation. The improved stability was maintained even on a scale of 200 kg (Table 13).

TABLE 13
Stability of a 200 kg Batch of 0.5% Carbomer Formula I

| Benzyl Alcohol | Crystals @ | Crystals @ | Crystals @ |
|---|---|---|---|
| (wt. %) | 5° C. | 10° C. | 15° C. |
| 3.0 | None 28 days | None 28 days | None 28 days |

Physical Stability of Carbomer Formula I vs. Metrogely Topical Gel. A physical stability study was conducted at 5° C. with Carbomer Formula I (3.0 wt. % benzyl alcohol and 0.5 wt. % carbomer) and Metrogel® 1% Topical Gel (Galderma). A placebo was prepared as a control. Results are shown below in Table 14.

TABLE 14
Physical Stability Results Carbomer
Formula I vs. Commercial Product

| | Crystals @ | Crystals @ |
|---|---|---|
| Sample | 5° C. | 5° C. |
| Carbomer Formula I | None 60 days | None 32 weeks |
| Metrogel ® Topical Gel | 48 days | 32 weeks: more |
| Lot 047112 (Exp August/2008) | | crystals than at 48 |
| | | days; larger crystals |
| | | than at 48 days |
| Placebo | None 60 days | None 32 weeks |

Carbomer Formula I provided increased stability compared to the commercial product, Metrogel® 1% Topical Gel. The commercial product gel showed metronidazole crystal formation when stored at 5° C. for just 48 days. Carbomer Formula I was stable at 5° C. for more than 32 weeks.

Based on the results described herein, the formulations containing 0.5% carbomer and 3.0% benzyl alcohol, and 1.25% HEC and 2.5% benzyl alcohol, as well as other formulations described herein, provide a significant improvement in stability over the commercial product with respect to the duration at which metronidazole can be maintained in the gel without crystal formation. Additionally, it was surprisingly discovered that this increase in stability can be provided in a variety of gel formulations, for example, Carbomer-based and HEC-based gel formulations that contain at least about 2.5 wt. % benzyl alcohol.

### Example 2

### Large Scale Preparation of 1% Metronidazole Gel

A 200 kg batch of 1% metronidazole gel with a carbomer gelling agent was prepared as follows. Percentages are w/w % of the final 200 kg batch. Purified water (88.5%; 177 kg) was added to an 80 gallon kettle equipped with a mixing apparatus (counter-rotating mixer or propeller mixer). The water was heated to about 40° C. and edetate disodium (0.05%; 0.1 kg), benzyl alcohol (3%; 6 kg), and metronidazole (1%; 2 kg) were added with stirring until the solids dissolved. The temperature was maintained, the stirring speed was increased to about 1200 RPM, and Carbomer 940 (0.5%; 1 kg) was slowly added until a homogeneous mixture formed, to provide Mixture A. Mixture A was stirred for an additional 45 minutes at about 40° C. Propylene glycol (3%; 6 kg; heated to about 40° C.) was added, followed by methylparaben (0.08%; 0.16 kg) and propylparaben (0.02%; 0.04 kg). Stirring was continued until the parabens dissolved. Mixing was continued at about 12 RPM, avoiding aeration, and the temperature was maintained for about 15-30 minutes to provide Mixture B.

Separately, purified water (0.4%; 0.8 kg) and sodium hydroxide (0.04%; 0.08 kg) were combined and mixed to homogeneity to provide Mixture C. Mixture C was slowly added to Mixture B with stirring, until completely dissolved. Stirring was continued at about 16 RPM for 20-30 minutes to provide a clear, smooth gel. The pH was measured (undiluted) and was found to be about 4.7. The pH can be adjusted by slow addition of a dilute aqueous 1.5% sodium hydroxide (heated to about 40° C.) if the pH is below about 4. Purified water (q.s. 100%; q.s. 200 kg) was added with stirring at about 14 RPM and was further stirred for about 45-60 minutes, at about 40° C., avoiding aeration. The batch was strained through a 40 mesh stainless steel screen directly into storage containers. The gel was stored in LDPE-lined stainless steel containers, which were covered to protect the composition from light.

All publications, patents, and patent documents are incorporated by reference herein, as though individually incorporated by reference. The invention has been described with reference to various embodiments and techniques. However, it should be understood that many variations and modifications may be made while remaining within the spirit and scope of the invention.

What is claimed is:

1. An aqueous gel composition comprising about 0.5 wt. % to about 2 wt. % metronidazole, about 0.5 wt. % to about 5 wt. % benzyl alcohol as a solvent, at least about 80 wt. % water, and a polyacrylic acid or cellulosic gelling agent present in about 0.25 wt. % to about 2 wt. %;
wherein the benzyl alcohol is present in an amount effective to maintain the physical stability of the aqueous gel composition for at least seven days at 5° C.

2. The gel composition of claim 1 wherein the metronidazole is present in about 1 wt. %.

3. The gel composition of claim 1 wherein the benzyl alcohol is present in about 1 wt. % to about 3.5 wt. %.

4. The gel composition of claim 1 wherein the benzyl alcohol is present in about 2.5 wt. % to about 3.0 wt. %.

5. The gel composition of claim 4 wherein the gel composition does not comprise a cosolvent other than benzyl alcohol.

6. The gel composition of claim 1 wherein the polyacrylic acid gelling agent is a carbomer gelling agent.

7. The gel composition of claim 6 wherein the carbomer gelling agent is present in about 0.5 wt. %.

8. The gel composition of claim 7 wherein the composition comprises about 2.5-3.5 wt. % benzyl alcohol and the composition does not include a further solubility enhancing agent.

9. The gel composition of claim 8 wherein the pH of the composition is about 4 to about 5.

10. The gel composition of claim 1 wherein the gelling agent is a polyacrylic acid and the viscosity is about 500 cps to about 32,000 cps.

11. The gel composition of claim 1 wherein the cellulosic gelling agent is a hydroxyalkyl cellulose gelling agent.

12. The gel composition of claim 11 wherein the hydroxyalkyl cellulose gelling agent is hydroxyethyl cellulose.

13. The gel composition of claim 12 wherein the composition comprises about 2-3 wt. % benzyl alcohol and the composition does not include a further solubility enhancing agent.

14. The gel composition of claim 13 wherein the hydroxyethyl cellulose is present in about 1 wt. % to about 1.5 wt. %.

15. The gel composition of claim 14 wherein the pH of the composition is about 5.3 to about 5.5.

16. The gel composition of claim 1 wherein the gelling agent is hydroxyethyl cellulose and the viscosity is about 500 cps to about 9,000 cps.

17. The gel composition of claim 1 wherein the benzyl alcohol is present in an amount effective to maintain the physical stability of the aqueous gel composition for at least 28 days at 5° C.

18. The gel composition of claim 1 wherein the benzyl alcohol is present in an amount effective to maintain the physical stability of the aqueous gel composition for at least 60 days at 5° C.

19. The gel composition of claim 1 wherein the benzyl alcohol is present in an amount effective to maintain the physical stability of the aqueous gel composition for at least 200 days at 5° C.

20. The gel composition of claim 1 wherein the gel is clear and colorless.

21. The gel composition of claim 1 comprising about 1% metronidazole, about 3% benzyl alcohol, about 0.5% Carbomer 940, about 3% propylene glycol, about 0.05% edetate disodium, about 0.1% parabens, about 0.04% sodium hydroxide, and at least about 90% water; wherein the benzyl alcohol is present in an amount that is effective to maintain the physical stability of the aqueous gel solution for at least seven days at 5° C.

22. The gel composition of claim 1 comprising about 1% metronidazole, about 2.5% benzyl alcohol, about 1.25% hydroxyethyl cellulose, about 3% propylene glycol, about 0.05% edetate disodium, about 0.1% parabens, and at least about 90% water; wherein the benzyl alcohol is present in an amount that is effective to maintain the physical stability of the aqueous gel solution for at least seven days at 5° C.

23. The gel of claim 1 wherein the composition is free of, or substantially free of, niacinamide, niacin, and cyclodextrins.

24. A method of treating a dermatologic disorder comprising topically applying to the site of the disorder an effective amount of an aqueous gel composition comprising about 0.5 wt. % to about 2 wt. % metronidazole, about 0.5 wt. % to about 5 wt. % benzyl alcohol as a solvent, at least about 80 wt. % water, and a polyacrylic acid or cellulosic gelling agent present in about 0.25 wt. % to about 2 wt. %;
wherein the benzyl alcohol is present in an amount effective to maintain the physical stability of the aqueous gel composition for at least seven days at 5° C.

25. The method of claim 24 wherein the application is once daily.

26. The method of claim 24 wherein the disorder comprises rosacea.

27. The method of claim 24 wherein the composition is free of, or substantially free of, niacinamide, niacin, and cyclodextrins.

28. A method for preparing an aqueous gel composition containing metronidazole comprising combining metronidazole, benzyl alcohol, and a gelling agent in an aqueous solution, wherein the gel composition contains benzyl alcohol at a concentration greater than about 0.5 wt. %, and the amount of benzyl alcohol in the resulting aqueous gel is sufficient to provide a dissolved concentration of metronidazole of greater than 0.5 wt. % at a temperature of 5° C. for at least about seven days.

29. A method for increasing the solubility of metronidazole in aqueous gel composition comprising combining metronidazole, benzyl alcohol, and a gelling agent in an aqueous fluid, wherein the amount of benzyl alcohol in the fluid is at least 0.5 wt. % and the amount of metronidazole in the fluid is at least about 0.75 wt. %.

30. The method of claim 29 wherein the benzyl alcohol is present in an amount effective to maintain the physical stability of the aqueous gel composition for at least about seven days at 5° C.
